(19) United States
(12) Patent Application Publication
(10) Pub. No.: US 2005/0042180 A1
(43) Pub. Date: Feb. 24, 2005

(54) Powder formulation containing the CGRP antagonist 1 [N2-[3,5-dibromo-N-[[4-(3,4-dihydro-2 (1H)-oxoquinazolin-3-yl)-1-piperidinyl]carbonyl]-D-tyrosyl]-L-lysyl]-4-(4-pyridinyl)-piperazin, process for preparing and the use thereof as inhalation powder

(75) Inventors: Michael Trunk, Ingelheim (DE); Claudius Weiler, Ingelheim-Grosswinterheim (DE); Christel Schmelzer, Ingelheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/909,624

(22) Filed: Aug. 2, 2004

Related U.S. Application Data
(60) Provisional application No. 60/503,015, filed on Sep. 15, 2003.

(30) Foreign Application Priority Data
Aug. 18, 2003 (DE) 103 38 403

(51) Int. Cl. A61K 38/04; A61K 9/14

(52) U.S. Cl. 424/46; 514/19; 514/252.17

(57) ABSTRACT

A powder inhalant for pulmonary or nasal inhalation, containing the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) in the form of spherically nanostructured microparticles, which are stable in their amorphous state under normal conditions (T<50° C., relative humidity <75%), a process for preparing these microparticles as well as the use thereof for preparing the powder inhalant for the treatment of headaches, migraine and cluster headache.

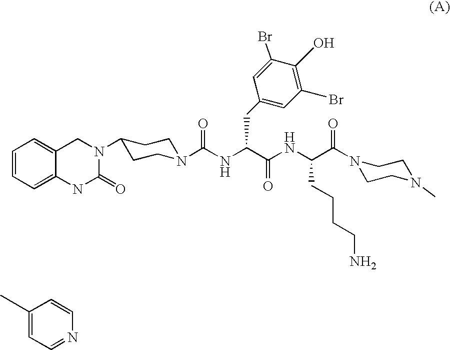

### RELATED APPLICATIONS

This application claims benefit of U.S. Ser. No. 60/503,015, filed Sep. 15, 2003, which is hereby incorporated by reference in its entirety.

### FIELD OF THE INVENTION

The invention relates to a powder inhalant for pulmonary or nasal inhalation, containing the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) in the form of spherically nanostructured microparticles, which are stable in their amorphous state under normal conditions (T<50° C., relative humidity <75%), a process for preparing these microparticles as well as the use thereof for preparing the powder inhalant for the treatment of headaches, migraine and cluster headache.

The spherically nanostructured microparticles according to the invention are suitable for preparing powder inhalants, in which no other excipients or additives (carrier materials) are needed in order to obtain an industrially workable powder which can be further processed directly and which has excellent properties in terms of dispersibility and is sufficiently easy to process with regard to its cohesive properties.

In another aspect the invention relates to the powder inhalants obtainable by the process according to the invention.

### BACKGROUND TO THE INVENTION

The CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) is known from International Patent Application PCT/EP97/04862 (published as WO 98/11128) and has the following structure:

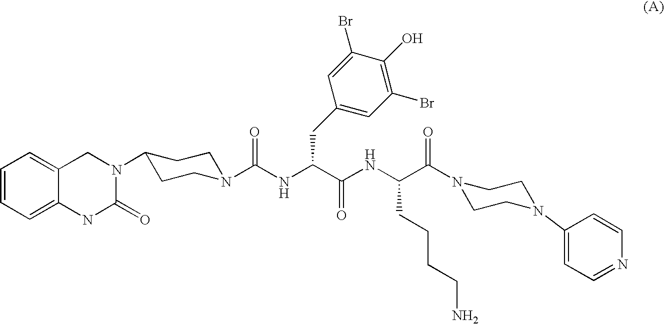

### PRIOR ART

The active substance base (A) is a highly effective CGRP antagonist for the acute and prophylactic treatment of headaches, particularly migraine and cluster headache, which cannot be administered orally using conventional formulations, as the substance has very limited oral bioavailability.

Moreover, the presumed therapeutic dose of this compound falls within a range which has not hitherto been technically possible for inhalants.

For treating sudden attacks of migraine it is essential that an active substance is systemically available as quickly as possible. The treatment should be uncomplicated for the patient to administer and no other conditions which could affect bioavailability (e.g. the food effect) should restrict the use of the medicament for the patient.

Active substances which are intended to be systemically available are usually administered by oral route. If this route is unsuitable or undesirable on account of particular properties of the active substance or particular demands made of the application, other possible ways of administering substances systemically are known in the art. For example, inhalation, by means of which active substances may be administered systemically as well as topically, has been under discussion for some time. For substances which prove critical on account of their decomposition in solution or which have poor solubility per se, powder inhalation is an option. The absolute amount of the active substance which has to be administered per application makes particular demands of the formulation. On the other hand, the physical stability (e.g. aerodynamic particle size, dispersibility, physicochemical properties) of the active substance has proved to be a critical requirement for the development and production of an inhalable powder.

With formulations of the powder inhalant type, inhalable powders, which are packaged for example in suitable capsules (inhalettes), are delivered to the lungs by means of powder inhalers. Similarly, other systems in which the quantity of powder to be administered is pre-dosed (e.g. blisters) and multidose powder systems are also known. Alternatively, the medicament may also be inhaled by the use of suitable powdered inhalable aerosols which are suspended for example in HFA134a, HFA227 or mixtures thereof as propellant gas.

In powder inhalation, the microparticles of a pure active substance are administered through the airways onto the surface of the lungs, e.g. in the alveoli, by the inhalation process. These particles settle on the surface and can only be absorbed into the body after the dissolving process by active and passive transporting processes.

Inhalation systems are known in the literature in which the active substance is present in the form of solid particles either as a micronised suspension in a suitable solvent system as carrier or in the form of a dry powder.

Usually, powder inhalants, e.g. in the form of capsules for inhalation, are prepared on the basis of the general teaching as described in DE-A-1 79 22 07.

A critical factor in multi-substance systems of this kind is the uniform distribution of the pharmaceutical composition in the powder mixture.

Another important aspect of powder inhalants is that when the active substance is administered by inhalation only particles of a certain aerodynamic size reach the target organ, the lungs. The average size of these lung-bound particles (inhalable fraction) is in the region of a few microns, typically between 0.1 und 10 μm, preferably less than 6 μm. Such particles are usually produced by micronisation (air-jet grinding).

It often happens that this mechanical step may cause particles of this kind to be complex in their composition in terms of their crystal properties. Also, the geometric shape of the particles of the starting material determines the morphological properties of the micronised preparation. For this formulation type it has proved important to use a thermodynamically stable or the most stable form of the active substance in powdered preparations of this kind. This is usually a crystalline form of the active substance.

It is known from the literature that particles in the submicron range can be produced by spray-drying. Usually, industrially suitable formulations which exhibit sufficient dispersibility in medical use (inhalation) may be prepared from spray-dried particles of this kind in accordance with the method cited above (DE-A-179 22 07) [Y.-F. Maa, P.-A. Ngyuyen, J. D. Andya, N. Dasovich, T. D. Sweeny, S. J. Shire, C. C. Hsu, Pharmaceutical Research, 15, No. 5 (1998), 768-775; M. T. Vidgrén, P. A. Vidgrén, T. P. Paronen, Int. J. Pharmaceutics, 35 (1987), 139-144; R. W. Niven, F. D. Lott, A. Y. Ip, J. M. Cribbs, Pharmaceutical Research, 11, No. 8 (1994), 1101-1109].

In these formulations as mentioned above, the uniform distribution of the pharmaceutical composition in the powder mixture is a critical factor, among others.

However, in addition to the requirements mentioned above, it should be borne in mind that powder inhalants in their general form are known primarily for topical use in diseases. The systemic administration of an active substance through the lungs is often discussed but it should be pointed out that the optimising of the actual bioavailability of an active substance is only inadequately described by the “lung-bound fraction of the active substance”, discussed in the literature and in the pharmacopoeias. This lung-bound fraction can be defined for example by means of the Andersen cascade impactor (corresponding to EP Suppl. 2002 or USP 25) as the proportion of particles less than 5 μm in size. A different way of looking at bioavailability depending on particle size is to discuss systemic use as a function of the active substance and its properties and to search for a solution which meets the requirements of the active substance.

In general terms the dependency of the particle size of the active substance which is made available for inhalation by powder administration was described by, among others, Köhler et al. [D. Köhler, W. Fleischer, “Theorie und Praxis der Inhalationstherapie”, Arcis-Verlag Munich 2000, page 25].

### STATEMENT OF THE PROBLEM

The complex aim of the invention was to provide an optimised spray-dried formulation of the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A), which meets the stringent requirements mentioned above that are imposed on a powder inhalant for pulmonary and nasal inhalation. The spray-dried formulation according to the invention, when compared with conventional micronised starting material (obtained e.g. by air-jet grinding), should prove suitable for use as a powder inhalant in terms of its pharmacological/pharmacokinetic properties. According to the invention the morphology of the microparticles was to be optimised so that the formulation consisting thereof preferably contains no excipient and hence consists exclusively of active substance.

The formulation according to the invention should also exhibit a rapid onset of activity for the treatment of the acute pain which occurs very suddenly in the case of migraine. This means that rapid absorption of the active substance and a rapid increase in the plasma level must be guaranteed.

### DETAILED DESCRIPTION OF THE INVENTION

A rapid onset of activity for the treatment of acute pain as well as a high plasma level of the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) and the physiologically acceptable salts thereof within a very short time can best be achieved through the lungs as the site of absorption. It has been found that when the active substance (A) is administered by inhalation in the form of a powder inhalant a bioavailability of about 60% can be achieved based on the fine content of the formulation (corresponding to FPD “fine particle dose”, determined according to USP 24 Suppl. 2000).

It has been found that the spray-dried formulation of the active substance base (A) according to the invention is particularly characterised in that it can make the active substance sufficiently systemically available for application by pulmonary or nasal inhalation, administering the smallest possible total amount (metered dose/nominal dose) of active substance per application.

The formulation is characterised by particular physicochemical properties of the microparticles linked to a nominal dose of active substance administered per application and requires no addition of carrier materials.

Surprisingly, it has been found that for systemic use of the active substance base (A), the fraction of particles having an aerodynamic size of less than 5 μm (measured using an Andersen cascade impactor) is not sufficient; rather, a greater fine fraction in which the particles have an aerodynamic size of less than 2.8 μm is needed for this substance.

The present invention describes suitable micronised powders having the necessary greater fine fraction, a method for producing these particles and some inhalette formulations by way of example.

In a first aspect the invention relates to a powder inhalant containing as active substance the active substance base 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) in the form of spherically nanostructured microparticles, characterised in that
(a) the particles have a specific surface area between 1 m²/g and 25 m²/g
(b) the characteristic value Q_{(5.8)} is between 50% and 100%,
(c) the parameter X₅₀ is in the range from 1 μm to 6 μm and
(d) the inhalable fine particle fraction below a particle size of 5 μm is greater than 40%, preferably the fraction below a particle size of 2.8 μm is greater than 20%, particularly preferably the fraction below a particle size of 2.8 μm is greater than 25%, based on the active substance content (metered dose) of the pharmaceutical composition.

These microparticles are characterised by special physical and physicochemical properties, which lead to an improved pharmacological/pharmacokinetic activity when the substance is administered by inhalation.

The availability of the substance—both quantitatively, based on the amount of active substance administered, and also in relation to achieving a high plasma level as quickly as possible—is determined not only by the biochemical properties of the substance but also by physicochemical properties. If a solid is administered—as in the case of a powder inhalant—the parameters of absolute solubility in the ambient medium and also speed of dissolving in the ambient medium as a function of the local concentration of the active substance and the time and also the site of deposition of the powder in the lungs (depending on the aerodynamic particle size) must be particularly taken into consideration.

Optimum administration by inhalation therefore has to take account of the fact that the particles of active substance form a finely divided coating on the surface of the lungs. The crucial factor here is that the active substance is changed such that the microparticles to be inhaled have advantages in terms of their particle-to-particle interaction and also their dispersing or aerodynamic properties which ensure that on the one hand they are deposited in quantity in the lower regions of the lungs and on the other hand the largest possible surface area of the lungs is covered. Therefore, the physical-chemical properties of the microparticles to be inhaled are of great importance in powder inhalants.

Particles produced by the process according to the invention are characterised by high physical stability. In particular, when used as a powder inhalant the particle properties allow a high fine content to be produced on delivery, technically measured by cascade impactor measurement, for example (Pharm Eur. 2002 “Inhalanda/Preparations for Inhalations” and USP 25<601> for dry powder inhalers, using an ANDERSEN 1 ACFM Mark II cascade impactor). Typically, the proportion of particles according to this method which are less than 5 μm in size (aerodynamically) is greater than 40%.

In addition to this key parameter for inhalants the powder is characterised in that it can be further processed directly by conventional industrial methods. Powders produced in this way are characterised by the physicochemical parameters of particle size, measured for example by laser diffraction, and specific surface area, measured for example by multipoint B.E.T. measurement. For the characteristic Q_{(5.8)} the particle size of powders produced in this way is typically between 50% and 100% and for the parameter X₅₀ in the range from 1 μm to 6 μm. Particles produced by the above methods typically have values for the specific surface area of between 1 m²/g and 25 m²/g, ideally between 1 m²/g and 20 m²/g, most preferably between 3 m²/g and 10 m²/g. Particularly suitable are powders according to the invention which have a characteristic value Q_{(2.5)} (corresponding to the quantity of particles which are less than 2.5 μm in size, based on the distribution by volume of the droplets) greater than 70%. In terms of geometry, particles prepared by the processes described above have particle shapes which may be described, depending on the test conditions, between the extremes of “spherical shape”, “spherical shape with cavity, possibly with hole”, “spherical shape with inwardly shaped convexities”, and “collapsed hollow body”. Under the scanning electron microscope the surface of such particles is substantially nanostructured.

It has been found according to the invention that the active substance base (A) can surprisingly be morphologically changed by a spray drying process so that a powder thus produced can be packed directly into a primary packaging means without any further steps, and above all without the need to mix it with a coarser carrier material, and can be delivered from this packaging for inhalation using a powder inhaler. The production process may be controlled so that the particles are of a suitable particle size, usually in the range from 0.1 μm to 10 μm, and these particles have surface properties such that they are easily vortexed/dispersed.

It has also been found that the particle morphology including the particle size can critically be controlled by the choice of process parameters and production parameters. A surprising fact is that powders of this substance which have been micronised using “conventional” jet-grinding processes and are present in a comparable range of particle sizes, still fundamentally differ morphologically from particles produced according to this invention, in terms of their surface properties/particle-to-particle interactions. This is apparent from the fact that the quality parameter “Fine Particle Fraction of Delivered Dose” (e.g. according to the method of determining the “Aerodynamic Particle Size Distribution”—Pharm Eur. 2002 “Inhalanda/Preparations for Inhalations” and USP 25<601> for dry powder inhalers) is improved by a factor 3 or more. As the use of a carrier material in the formulation is unnecessary, the absolute dose of active substance actually available to the patient improves by an even higher factor for a given total amount of powder to be administered.

The method of preparation according to the invention is characterised in that the active substance is suitably dissolved, sprayed and dried in a spray tower. The principle of spray drying consists in dividing a solution or suspension of the product which is to be dried into fine droplets and drying them with a stream of hot gas. The solid fraction remaining after evaporation of the solvent is separated from the gas current by means of a gravity separator (e.g. a cyclone) and/or a filter unit and collected. The microparticles thus produced are characterised by special values in terms of particle size, specific surface area and morphology.

A micronised preparation thus obtained may be used directly as a powder inhalant. There is no need to mix it with a coarser carrier material. The quantity of powder to be administered is suitably presented to the patient in the form of a premetered dose. This may be done, for example, by packing 10 mg to 100 mg, preferably 20 mg to 60 mg of this micronised preparation obtained by the above method into inhalettes (or into some other suitable form, e.g. blisters, from which it can be inhaled using suitable inhalers) and administered using a Handihaler®, for example.

Organic solvents, organic-aqueous solvent mixtures have proved suitable as solvents for preparing the required microparticles by spray-drying. Preferably an alcoholic-aqueous solvent system is used, particularly preferably a solvent mixture consisting of ethanol/methanol/water or ethanol/propanol/water and most particularly preferably the solvent mixture of ethanol/water. The molar proportion of water in the solvent mixtures should be set at 0.1 to 10 times the amount of the molar proportion of the alcohol components, preferably 0.5 to 4 times the amount. Adjusting the concentration of active substance serves primarily to make the process economical. However, limits are set on the concentration of active substance to be achieved, which are prescribed by the fact that the surface qualities of the particles can be optimised by achieving a specific ratio between the droplet size and concentration of solids and a certain particle size is obtained depending on the droplet size and the solids concentration. Usually, a concentration of 0.5 wt. % to 20 wt. %, preferably from 2 wt. % to 10 wt. %, particularly preferably from 2.5 wt. % to 7 wt. %, should be selected.

The droplet size is a crucial parameter in the production of inhalable particles. Depending on the nozzle used the throughput of spray gas in conjunction with the throughput of solution should be selected so as to obtain the desired droplet size. As there are a number of combinations of the parameters nozzle-spray gas throughput-solution throughput which lead to a suitable droplet size, the process can usefully be defined by means of the droplet size selected for the process. This may be characterised by the parameter X₅₀ (median value=particle size/droplet size, below which 50% of the quantity of particles fall, based on the distribution by volume of the individual particles/droplets), which should be in the range from 1 μm to 20 μm, preferably from 1 μm to 8 μm, particularly preferably from 1 μm to 3 μm, and by the characteristic Q_{(5.8)} (corresponding to the quantity of particles which fall below 5.8 μm, based on the distribution by volume of the droplets), which should be between 10% and 100%, preferably between 30% and 100%, particularly preferably between 60% and 100%.

This is implemented technically by using a corresponding commercial nozzle, e.g. a single- or multi-substance nozzle, which has these characteristics depending on the nozzle parameters (e.g. the speed of rotation with rotary atomisers or the atomising pressure applied and the resulting mass flow of the atomising gas in the case of two-substance nozzles) and the spray rate (flow volume of “spray solution”). Besides the special conditions which have to be adhered to during the actual spraying process in order to generate droplets which are suitable for the drying process, it is found that the surface properties of the particles can also be positively/deliberately influenced by the choice of drying parameters. The critical characteristics which impinge on the drying step are the entry and exit temperature of the drying gas, as well as the flow volume of the drying gas passed through. Care must be taken to ensure that the drops of suitable droplet size are passed through the drying chamber in such a way that the droplets and the dried particles make little or no contact with the wall of the spraying tower. This is achieved by using nozzles with a suitable spray cone, a spraying tower of suitable diameter and by means of the flow conditions in the apparatus. The starting temperature must be adapted to the process so that the powder has a low enough residual solvent content and hence adequate chemical and physical stability is achieved. This is ideally done by keeping the exit temperature in the region of the boiling temperature or slightly above. On the other hand, the inlet temperature of the drying gas should be selected so that in conjunction with the parameter of the flow volume of the drying gas and the spray rate the drying is gentle enough to form particles with suitable surface properties. The spray drying process must also be designed so that the fine particles described above can also be recovered. This may be done for example using suitable cyclones or very fine particle filters.

In a second aspect the invention thus relates to a process for preparing the active substance base (A) in the form of spherically nanostructured microparticles, comprising the steps of
(a) dissolving the active substance base (A) in an organic solvent or an organic-aqueous solvent mixture to prepare a solution of the active substance with a concentration of active substance of 0.5 wt. % to 20 wt. %, preferably from 2 wt. % to 10 wt. %, particularly preferably from 2.5 wt. % to 7 wt. %,
(b) spraying the active substance solution thus obtained in the usual way, so as to obtain a spray mist with a droplet size having the parameter X₅₀ in the range from 1 μm to 20 μm, preferably 1 μm to 8 μm, particularly preferably 1 μm to 3 μm, and the characteristic value Q_{(5.8)} between 10% and 100% (measured by Sympatec laser diffraction), preferably between 30% and 100%, particularly preferably between 60% and 100%,
(c) drying the spray mist thus obtained using a drying gas while adhering to the following parameters:
   (i) an entry temperature of the drying gas of from 100° C. to 350° C., preferably from 120° C. to 250° C., particularly preferably from 130° C. to 200° C., and
   (ii) an exit temperature of the drying gas of from 40° C. to 120° C. and
(d) separating the dried solid fraction from the current of drying gas in the usual way.

A preferred process according to the invention is a process for preparing the active substance base (A) in the form of spherically nanostructured microparticles, comprising the steps of
(a) dissolving the active substance base (A) in an organic solvent or an organic-aqueous solvent mixture for preparing a solution of the active substance with a concentration of active substance of 0.5 wt. % to 20 wt. %, preferably from 2 wt. % to 10 wt. %, particularly preferably from 2.5 wt. % to 7 wt. %,
(b) spraying the active substance solution thus obtained in the usual way with a flow volume of the spray gas from 1 Nm³/h to 15 Nm³/h, preferably from 3 Nm³/h to 15 Nm³/h, so as to obtain a spray mist with a droplet size having the parameter X₅₀ in the range from 1 μm to 20 μm, preferably from 1 μm to 8 μm, particularly preferably from 1 μm to 3 μm, and the parameter Q_{(5.8)} between 10% and 100% (measured by Sympatec laser diffraction), preferably between 30% and 100%, particularly preferably between 60% and 100%,
(c) drying the spray mist thus obtained using a drying gas while adhering to the following parameters:
   (i) an entry temperature of the drying gas from 100° C. to 350° C., preferably from 120° C. to 250° C., particularly preferably from 130° C. to 200° C.,
   (ii) an exit temperature of the drying gas from 40° C. to 120° C. and (iii) a flow volume of the drying gas from 15 Nm³/h to 1500 Nm³/h, preferably from 15 Nm³/h to 150 Nm³/h, and
(d) separating the dried solid fraction from the current of drying gas in the usual way.

In an alternative to the above processes the micronised preparation may be homogeneously mixed with an excipient by conventional methods before the filling operation.

Micronised preparations obtained as above may be administered directly by inhalation (but also in the form of a powder mixture). For this, the quantity of powder to be administered must be pre-dosed into an inhalation capsule. The powder may be administered using an inhaler, e.g. the Handihaler®. Alternatively, a single dose of this micronised preparation may also be prepared in comparable manner in the form of e.g. blister wells. A blister may be placed in an inhaler (device) and during the inhalation process the single dose is emptied into the device from the wells or the powder is inhaled directly therefrom. It is also possible for powdered formulations to be administered using a multi-dose powder inhaler. In principle, the quantity of powder to be inhaled from a storage container is divided up before application, and in a second step this dose of powder is inhaled. Surprisingly, it has been found that the dispersal of the amorphous micronised preparation obtained by spray drying takes place from the primary packaging without any problems and the aerodynamic particle size distribution meets the requirements for the systemic administration of the active substance. It has been found that in this way powder inhalants consisting of the pure active substance micronisate can be prepared which have a FPF<5 μm (measured using the Andersen cascade impactor) of >40%. In particular, these powder inhalants are characterised by a fine particle fraction of the FPF with a size of separation <2.8 μm of more than 20%, preferably more than 25%.

Surprisingly, the powder inhalants prepared from this active substance as described above are characterised in that the micronised preparations are present in amorphous form and can be used irrespective of the increased humidity and temperature. The products described here have a breaking stability with regard to the above physical/aerodynamic properties of several days under climatic conditions of 40° C. and 75% relative humidity.

The invention described herein therefore also relates to premetered inhalable powders, particularly inhalation capsules or blister wells filled with micronised preparation, which can be administered using inhalers. The capsules provided in this form for application or quantities of powder packaged in other ways contain amorphous micronised active substance in an amount of from 10 mg to 100 mg, preferably from 15 mg to 70 mg, particularly preferably from 20 mg to 60 mg.

In a third aspect the present invention relates to a powder inhalant according to the invention which can be obtained by a process as hereinbefore described.

In a fourth aspect the present invention relates to the use of the active substance base (A) in the form of the spherically nanostructured microparticles, which can be obtained by a process as hereinbefore described, for preparing a powder inhalant for pulmonary or nasal inhalation.

In a fifth aspect the present invention relates to a premetered pharmaceutical form containing a powder inhalant according to the invention which contains an amount of active substance in the range from 10 mg to 100 mg, preferably from 15 mg to 70 mg, particularly preferably from 20 mg to 60 mg.

A sixth aspect relates to a capsule for inhalation (powder inhalette), containing a powder inhalant according to the invention which contains an amount of active substance in the range from 10 mg to 100 mg, preferably from 15 mg to 70 mg, particularly preferably from 20 mg to 60 mg.

### EXPERIMENTAL SECTION

1) Methods of Measurement

a) Determining the Particle Size by Laser Diffraction (Frauenhofer Diffraction):

Measuring method: In order to determine the particle size the powder is fed into a laser diffraction spectrometer using a dispersing unit. The median value X₅₀ refers to the particle size below which 50% of the quantity of particles fall. The Q_{(5.8)} value describes the percentage of particles which are less than 5.8 μm in size. The Q_{(2.5)} value describes the percentage of particles which are less than 2.5 μm in size.
Measuring device: Laser diffraction spectrometer (HELOS), Messrs. Sympatec
Software: WINDOX Version 4
Dispersing unit: RODOS/dispersing pressure: 3 bar
Focal length: 50 mm [measuring range: 0.45 . . . 87.5 μm]
Evaluation method: HRLD (V 3.3 Rel. 1)
   b) Determining the Specific Surface Area:
Measuring method: The specific surface area is determined by exposing the powder sample to a nitrogen atmosphere at different pressures. Cooling the sample causes the nitrogen molecules to be condensed on the surface of the particles. The quantity of condensed nitrogen is determined by means of the drop in pressure in the system and the specific surface area of the sample is calculated by means of the surface nitrogen requirement and the weight of the sample.
Measuring device: Tri Star Multi Point BET, Messrs. Micromeritics
Heating station: VacPrep 061, Messrs. Micromeritics

Heating: approx. 12 h/40° C.

| Analysis parameters | |
|---|---|
| sample tube: | ½ inch; with filler rod |
| analysis method: | 16 point BET surface measurement |
| | 0.05 to 0.20 p/p0 |
| absolute pressure tolerance: | 5.0 mm Hg |
| relative pressure tolerance: | 5.0% |
| evacuation rate: | 50.0 mm Hg/second |
| evacuation threshold: | 10.0 mm Hg |
| evacuation time: | 0.1 h |
| free space: | lower Dewar, t: 0.5 h |
| retention time: | 20 seconds |
| minimum equilibration delay: | 600 seconds |
| adsorptive: | nitrogen |

c) Determining the Droplet Size by Laser Diffraction (According to Mie):
Measuring device: Laser diffraction spectrometer (HELOS), Messrs. Sympatec
Software: WINDOX Version 4
Focal length: 100 mm [measuring range: 0.9 . . . 175 μm]
Measuring method: The droplet size is determined by removing the nozzle from the spray dryer and placing the spray in the upper third of the spray cone in the centre of the laser beam. Measuring is done at ambient temperature with water as reference medium under otherwise identical conditions.

d) Determining the Aerodynamic Particle Size

| | |
|---|---|
| Method: | according to Pharm Eur. 2002 “Inhalanda/Preparations |
| | for Inhalations” and USP 25<601> for dry powder |
| | inhalers |
| Impactor: | ANDERSEN 1 ACFM Mark II cascade impactor (8 |
| | stages, end filter and preseparator, USP high top, |
| | sample induction port (SIP) |
| Inhaler: | Handihaler ® |
| flow volume: | 39 L/min |
| FPF (<5 μm): | Deposition plates 2-7 incl. filter |
| FPF (<2.8 μm): | Deposition plates 4-7 incl. filter |

### 2) EXAMPLES

### Example 1

### Spray Parameters Suitable for an Alcoholic Solution of (A) (Modified BÜCHI Spraying Tower)

| | |
|---|---|
| Concentration of solution/ | 4.0 g (A)/100 g ethanol/H₂O |
| Composition of solvent | molar ratio: 2:3 |
| Droplet size Q_{(5.8)} | 59% |
| (Reference solution: H₂O at ambient | |
| temperature) | |
| X₅₀ | 6.5 μm |
| flow volume “spray rate” | 1.2 L/h |
| Spray pressure (nozzle type) | 5.5 bar overpressure (N₂) |
| | (BÜCHI spray nozzle 0.7 mm, |
| | Art.-No. 04364) |
| Mass flow of spray gas (nozzle type) | 3.4 kg/h |
| | (BÜCHI spray nozzle 0.7 mm, |
| | Art.-No. 04364) |
| entry temperature | 150° C. |
| exit temperature | 100° C. |
| flow volume “drying gas” | 36 Norm m³/h |
| cross section of drying tower | 105 mm |

3) Characterisation of the Solid Particles Obtained

| | | |
|---|---|---|
| particle size | X₅₀ | 1.6 μm |
| | Q_{(2.5)} | 82.5% |
| | Q_{(5.8)} | 98.9% |
| specific surface area: | Sₘ | 7.5 m²/g |

1. A pPowder inhalant, comprising the active substance base 1-[/-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine of formula 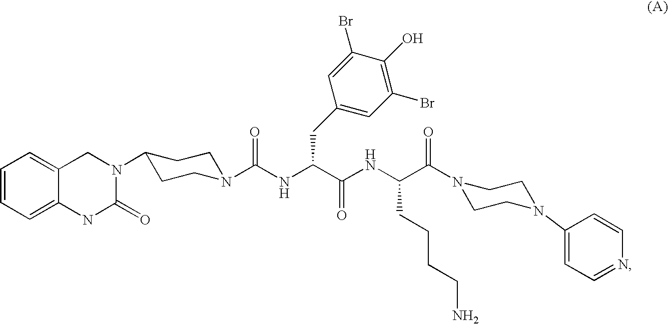
in the form of spherically nanostructured microparticles, characterised in that
(a) the particles have a specific surface area between 1 m²/g and 25 m²/g, preferably between 1 m²/g and 20 m²/g, particularly preferably between 3 m²/g and 10 m²/g,
(b) the characteristic value Q_{(5.8)} is between 50% and 100%,
(c) the parameter X₅₀ is in the range from 1 μm to 6 μm and (d) the inhalable fine particle fraction below a particle size of 5 μm is greater than 40%, based on the active substance fraction (the metered dose) of the pharmaceutical composition.

2. The powder inhalant according to claim 1, characterised in that the characteristic value Q_{(2.5)} is between 70% and 100%.

3. The powder inhalant according to claim 1, characterised in that it contains a fine particle fraction below a particle size of 2.8 μm of more than 20%.

4. The powder inhalant according to claim 1, characterised in that it contains a fine particle fraction below a particle size of 2.8 μm of more than 25%.

5. A process for preparing the active substance base 1-[/N²-[3,5-dibromo-N-[[4-(3,4-dihydro-2(1-oxoquinazolin-3-yl)-1-piperidinyl]carbonyl]-D-tyrosyl]-L-lysyl]-4-(4-pyridinyl)-piperazine of formula 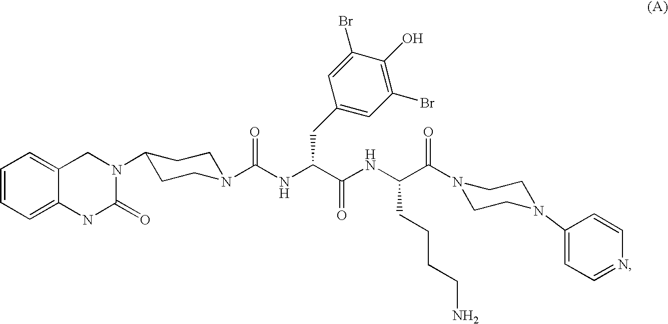
in the form of spherically nanostructured microparticles, comprising the steps of
(a) dissolving the active substance base (A) in an organic solvent or an organic-aqueous solvent mixture to prepare a solution of the active substance with a concentration of active substance of 0.5 wt. % to 20 wt. %, preferably from 2 wt. % to 10 wt. %, particularly preferably from 2.5 wt. % to 7 wt. %,
(b) spraying the active substance solution thus obtained in the usual way, so as to obtain a spray mist with a droplet size having the parameter X₅₀ in the range from 1 μm to 20 μm, preferably from 1 μm to 8 μm, particularly preferably from 1 μm to 3 μm, and the characteristic value Q_{(5.8)} between 10% and 100% (measured by Sympatec laser diffraction), preferably between 30% and 100%, particularly preferably between 60% and 100%,
(c) drying the spray mist thus obtained using a drying gas, while adhering to the following parameters:
(i) an entry temperature of the drying gas of from 100° C. to 350° C., preferably from 120° C. to 250° C., particularly preferably from 130° C. to 200° C., and
(ii) an exit temperature of the drying gas of from 40° C. to 120° C. and
(d) separating the dried solid fraction from the current of drying gas in the usual way.

6. A process for preparing the active substance base (A), 1-[N²-3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine of formula 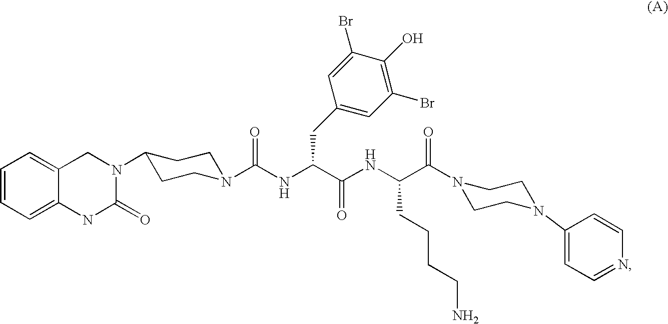
in the form of spherically nanostructured microparticles, comprising the steps of
(a) dissolving the active substance base (A) in an organic solvent or an organic-aqueous solvent mixture to prepare a solution of the active substance with a concentration of active substance of 0.5 wt. % to 20 wt. %, preferably from 2 wt. % to 10 wt. %, particularly preferably from 2.5 wt. % to 7 wt. %,
(b) spraying the active substance solution thus obtained in the usual way with a flow volume of the spray gas from 1 Nm³/h to 15 Nm³/h, preferably from 3 Nm³/h to 15 Nm³/h, so as to obtain a spray mist with a droplet size having the parameter X₅₀ in the range from 1 μm to 20 μm, preferably from 1 μm to 8 μm, particularly preferably from 1 μm to 3 μm, and the parameter Q_{(5.8)} between 10% and 100% (measured by Sympatec laser diffraction), preferably between 30% and 100%, particularly preferably between 60% and 100%,
(c) drying the spray mist thus obtained using a drying gas while adhering to the following parameters:
(i) an entry temperature of the drying gas from 100° C. to 350° C., preferably from 120° C. to 250° C., particularly preferably from 130° C. to 200° C.,
(ii) an exit temperature of the drying gas from 40° C. to 120° C. and
(iii) a flow volume of the drying gas from 15 Nm³/h to 1500 Nm³/h, preferably from 15 Nm³/h to 150 Nm³/h, and
(d) separating the dried solid fraction from the current of drying gas in the usual way.

7. The process according to claim 5 or 6, characterised in that the solvent used to dissolve the active substance is an organic-aqueous solvent system, the molar proportion of the water being from 0.1 to 10 times the amount of the molar proportion of the alcohol components, preferably from 0.5 to 4 times the amount.

8. The process according to claim 5 or 6, characterised in that the organic-aqueous solvent system consists of ethanol/methanol/water, the molar proportion of the water being from 0.1 to 10 times the amount of the molar proportion of the alcohol components, preferably from 0.5 to 4 times the amount.

9. The process according to claim 5 or 6, characterised in that the organic-aqueous solvent system consists of ethanol/propanol/water, the molar proportion of the water being from 0.1 to 10 times the amount of the molar proportion of the alcohol components, preferably from 0.5 to 4 times the amount.

10. The process according to claim 5 or 6, characterised in that the organic-aqueous solvent system consists of ethanol/water, the molar proportion of the water being from 0.1 to 10 times the amount of the molar proportion of the alcohol components, preferably from 0.5 to 4 times the amount.

11. The product of the process of claim 5, 6, 7, 8 9 or 10.

12. A premetered pharmaceutical form, containing a powder inhalant according to one of claims 1 to 4 or the product of the process of claim 5, 6, 7, 8, 9 or 10, which has a content of active substance in the range from 10 to 100 mg, preferably from 15 mg to 70 mg, particularly preferably from 20 mg to 60 mg.

13. A capsule for inhalation (powder inhalette), containing a powder inhalant according to one of claims 1 to 4 or the product of the process of claim 5, 6, 7, 8, 9 or 10, which has a content of active substance in the range from 10 to 100 mg, preferably from 15 mg to 70 mg, particularly preferably from 20 mg to 60 mg.
